(19) United States
(12) Patent Application Publication
(10) Pub. No.: US 2005/0214883 A1
(43) Pub. Date: Sep. 29, 2005

(54) Novel alanine transaminase enzyme and methods of use

(76) Inventors: Da-Wei Gong, Olney, MD (US); Alan Shuldiner, Columbia, MD (US); Rongze Yang, Baltimore, MD (US)

Correspondence Address: Pauley Petersen & Erickson, Suite 365, 2800 W. Higgins Road, Hoffman Estates, IL, 60195, US

(21) Appl. No.: 11/126,421

(22) Filed: May 11, 2005

Related U.S. Application Data
(62) Division of application No. 10/477,086, filed on Nov. 6, 2003.
(60) Provisional application No. 60/290,829, filed on May 14, 2001.

(51) Int. Cl. G01N 33/53; G01N 33/537; G01N 33/543

(52) U.S. Cl. 435/7.93

(57) ABSTRACT

A novel alanine transaminase gene (ALT2) is isolated from human tissue. ALT2 specific polynucleotides, polypeptides, and antibodies are described. ALT2 is expressed predominately in liver, kidney, brain, muscle, and adipose tissue. ALT2 can be used to diagnose injury and diseases involving tissues expressing ALT2.

This application claims priority to U.S. Patent Application 60/290,829 filed on May 14, 2001, which is hereby incorporated in its entirety by reference.

This invention was supported by grant 1 R21 DK57835-01 from the National Institute of Diabetes and Digestive and Kidney Diseases, National Institutes of Health. The U.S. Government has certain rights in this invention.

### BACKGROUND OF THE INVENTION

1. Field of the Invention

The present invention relates to a novel homolog of human alanine transaminase (ALT), ALT2, and the use of ALT2 as a diagnostic marker to predict and monitor tissue damage and/or tissue malfunction. The present invention relates to assays for ALT2 to diagnose tissue damage and/or tissue malfunction having a range of etiologies that include but are not limited to hepatitis, nonalcoholic steatohepatitis (NASH), fatty liver, cirrhosis, and drug hepatotoxicity, and other disorders in muscle, brain, kidney and adipose tissue.

2. Related Art

Alanine transaminase (ALT) [EC 2.6.1.2., also called glutamate pyruvate transaminase (GPT) and alanine aminotransferase] is a pyridoxal enzyme catalyzing reversible transamination between alanine and 2-oxoglutarate to form pyruvate and glutamate. ALT activity is present in many tissues including liver, muscle, heart, kidney, and brain. By mediating the conversion of these four major intermediate metabolites, ALT plays an important role in gluconeogenesis and amino acid metabolism. In muscle and certain other tissues that degrade amino acids for fuel, amino groups are collected from glutamate by transamination. ALT transfers the α-amino group from glutamate to pyruvate to form alanine, which is a major amino acid in blood during fasting. Alanine is taken up by the liver for generating glucose from pyruvate in a reversal ALT reaction, constituting the so-called alanine-glucose cycle (Felig, The glucose-alanine cycle, Metabolism 22:179-207 (1973)). This cycle is also important during intensive exercise when skeletal muscles operate anaerobically, producing not only ammonia groups from protein breakdown but also large amounts of pyruvate from glycolysis. In addition, alanine transamination was reported to be important for generating glutamate, an important neurotransmitter in the brain (Peng, et al., Utilization of alpha-ketoglutarate as a precursor for transmitter glutamine in cultured cerebellar granule cells, Neurochem. Res. 16:29-34 (1991)). In cultured cerebellar granule cells, 2-oxoglutarate combined with an amino group donor (alanine) is a precursor for the glutamate pool that is released by potassium-induced depolarization. The transamination inhibitor aminooxyacetic acid inhibits the formation of glutamate, indicating that ALT participates in the synthesis of this important inhibitory neurotransmitter.

ALT is used clinically as a surrogate marker for liver function. Serum ALT is significantly elevated when the liver is damaged by drug toxicity, infection (bacterial, viral, fungal, protozoan or other eukaryotic organism), alcohol, and steatosis (Dufour, et al., Diagnosis and monitoring of hepatic injury. II. Recommendations for use for laboratory tests in screening, diagnosis, and monitoring, Clin. Chem. 46: 2050-2068 (2000); Sherman, K. E., Alanine aminotransferase in clinical practice, Arch. Intent. Med. 151:260-265 (1991)).

While low level of ALT is present in peripheral circulation because of normal cell turnover or release from nonvascular sources, the liver contains the highest levels of ALT (Pratt and Kaplan, supra). The difference between ALT levels in liver and in blood is about 2,000-3,000-fold (Lott, J. A., Alanine and aspartate aminotransferase (ALT and AST), Year Book Medical Publisher, Chicago, 111-138 (1986)). Hence, the increased ALT in serum, plasma, or blood is regarded as a marker of liver injury because of the “leakage” of hepatic ALT into the circulation. Usually, the nature of liver injury cause the blood ALT levels to vary greatly (Dufour, et al., Diagnosis and monitoring of hepatic injury. I. Performance characteristics of laboratory tests, Clin. Chem., 46:2027-2049 (2000); Dufour, II, supra; and Sherman, supra). Extremely high transaminase levels (>8- to 10-fold normal) may indicate acute viral hepatitis and drug-induced hepatoxicity. A mild chronic increase of serum ALT (2- to 8-fold) is characteristic of chronic hepatitis, fatty liver and steatosis. However, the mechanism for the correlation of ALT levels with the etiology of liver damage remains to be understood.

Even though serum ALT is one of the most widely-used assays in clinical chemistry, there are serious deficiencies with the assay because it is an inadequate predictor in some cases. Recent studies cast doubt on serum ALT assay's specificity for liver disease. Higher than normal ALT levels are frequently associated with other clinical conditions such as obesity, muscle disease, heart failure, hemochromatosis, Wilson's disease, and α1-antitrypsin deficiency (see, Asayama, et al., Relationships between an index of body fat distribution (based on waist and hip circumferences) and stature, and biochemical complications in obese children, Int. J. Obes. Relat. Metab. Disord., 22:1209-16 (1998); Strauss, et al., Prevalence of abnormal serum aminotransferase values in overweight and obese adolescents [see comments], J. Pediatr. 136:727-33 (2000); Rutledge, et al., Persistent hypertransaminasemia as the presenting finding of childhood muscle disease, Clin. Pediatr. (Phila)., 24:500-503 (1985); Lin, et al., Persistent hypertransaminasemia as the presenting findings of muscular dystrophy in childhood, Taiwan Erh Ko I Hsueh Hui Tsa Chih. 40:424-429 (1999); Lott and Landesman, The enzymology of skeletal muscle disorders, Crit. Rev. Clin. Lab. Sci., 20:153-190 (1984); Friedman, et al., Evaluation of blood donors with elevated serum alanine aminotransferase levels, Ann. Intern. Med., 107:137-44 (1987); and Lozano, et al., Study of serum alanine-aminotransferase levels in blood donors in Spain, Haematologica, 83:237-239 (1998)). Higher than normal levels of ALT are also observed in many asymptomatic or “healthy” patients because ALT levels are influenced by age, gender, diet, and drugs (Sherman, supra; Pratt and Kaplan, Evaluation of abnormal liver-enzyme results in asymptomatic patients, N. Engl. J. Med., 342:1266-1271 (2000); and Blanc and Redlich; Elevated liver enzymes in asymptomatic patients, N. Engl. J. Med., 343:662; discussion 663 (2000)).

The existence of ALT isoenzymes has been suspected, but nobody has previously isolated the different isoforms. Early work indicated that there are two ALT activities, cytosolic and mitochondrial, separable by chromatography (Ziegenbein, R., Two different forms of glutamic pyruvic transaminase in rat heart and their intracellular localization, Nature, 212:935 (1966)). Recent literature generally regards ALT as a cytosolic enzyme (Asayama, et al., Relationships between an index of body fat distribution (based on waist and hip circumferences) and stature, and biochemical complications in obese children, Int. J. Obes. Relat. Metab. Disord., 22:1209-1216 (1998)). Nevertheless, both biochemical and cytogenetic studies have suggested the existence of two ALT isoforms in humans. Using classical chromatography, human liver, kidney, and skeletal and cardiac muscles have both cytosol and mitochodrial ALT activities with different biochemical kinetics (Gubern, et al., Partial characterization of the alanine aminotransferase isoenzymes from human liver, Biochem. Soc. Trans., 18: 1288-1289 (1990); and Sakagishi, Y., [Alanine aminotransferase (ALT)], Nippon Rinsho, 53:1146-1150 (1995)). Using a cytogenetic approach, Kielty et al., studied segregation in hybrids made from a rat hepatoma cell line and various human cells of nonhepatic origin, and mapped the cytoplasmic hepatic form of ALT to chromosome 8 (Kielty, et al., Regulation of expression of liver-specific enzymes. II. Activation and chromosomal localization of soluble glutamate-pyruvate transaminase, Ann. Hum. Genet., 46:135-143 (1982)). On the other hand, Wijnen and Meera Khan reported that cytosolic ALT is on chromosome 16 by studying hybrids between human leukocytes and Chinese hamster fibroblasts (Wijnen, Assignment of GPT to human chromosome 16, Cytogenet. Cell Genet. 32:327 (1982)). More recently, based on the peptide sequence of a liver cytosolic ALT (Ishiguro, et al., Complete amino acid sequence of human liver cytosolic alanine aminotransferase (GPT) determined by a combination of conventional and mass spectral methods, Biochemistry, 30:10451-10457 (1991)), Sohocki et al., cloned a human ALT gene, (which is designated ALT1 for the purposes of this invention), and mapped it to human chromosome 8 (Sohocki, et al., Human glutamate pyruvate transaminase (GPT): localization to 8q24.3, cDNA and genomic sequences, and polymorphic sites, Genomics, 40:247-252 (1997)).

Information relevant to the production and detection of ALT can be found in U.S. Pat. Nos. 5,952,211 and 5,804,402. However, neither one of these references describes a naturally-occurring isoenzyme of human ALT which is likely to offer improvements over currently used diagnostic assays for liver damage.

### SUMMARY OF THE INVENTION

It is an object of this invention to have an ALT2 polypeptide which has the amino acid sequence of SEQ ID NO: 2.

It is another object of this invention to have a polynucleotide which encodes the ALT2 polypeptide. It is a further object of this invention that the polynucleotide encodes the amino acid sequence of SEQ ID NO: 2 or a homolog of SEQ ID NO: 2. It is also further object of this invention that the polynucleotide sequence be the sequence of SEQ ID NO: 1.

It is another object of this invention to have an antibody which binds specifically to ALT2. This antibody is specific for ALT2 and does not bind to ALT1. Furthermore, this antibody can bind to the ALT2 sequence of SEQ ID NO: 2 or an ALT2-specific fragment thereof or a homolog of SEQ ID NO: 2, or, alternatively, to the protein encoded by the DNA sequence of SEQ ID NO: 1 or an ALT2-specific fragment thereof.

It is an object of this invention to have an expression vector for ALT2. This expression vector can be a plasmid, cosmid, or other type of vector where the DNA sequence encoding ALT2 is operatively linked to expression sequences, such as a promoter. The DNA sequence for ALT2 can be the sequence in SEQ ID NO: 1 or can be a sequence which encodes for the amino acid sequence of SEQ ID NO: 2, or a homolog of SEQ ID NO: 2.

It is an object of this invention to have a method for detecting the presence of ALT2 mRNA and/or ALT1 mRNA in a sample. It is a further object of this invention that the sample can be tissue or bodily fluids from an animal, including but not limited to human and mammals. It is a further object of this invention that a polynucleotide probe be used to detect the presence of ALT2 mRNA and/or ALT1 mRNA in a sample.

It is an object of this invention to have a method to detect the presence of ALT2 protein and/or ALT1 protein in a sample. It is a further object of this invention that the sample can be tissue or bodily fluids from an animal, including but not limited to human and mammals. It is another object of this invention that one uses antibodies (monoclonal or polyclonal) that bind specifically to ALT2 or that bind specifically to ALT1 to detect the respective protein. It is another object of this invention that the bodily fluids can be blood, serum, lymph, urine, sweat, mucus, sputum, saliva, semen, spinal fluid, interstitial fluid, synovial fluid, cerebrospinal fluid, gingival fluid, vaginal fluid, and pleural fluid. It is also an object of this invention that the tissue can be liver, brain, muscle, adipose tissue, and kidney.

It is another object of this invention to have a method for diagnosing or detecting injury or disease involving tissue which contains ALT2. It is a further object of this invention that the method involves using antibodies (polyclonal or monoclonal) that specifically bind to ALT2 to measure the level of ALT2 in bodily fluids from the animal. It is another object of this invention to use antibodies (polyclonal or monoclonal) that specifically bind to ALT1 to measure the level of ALT1 in bodily fluids from the animal and then to compare the level of ALT2 to ALT 1. When the level of ALT2 is sufficiently higher than the level of ALT1 or the level of ALT2 falls within a predetermined range, then the animal is diagnosed with a specific disease or injury. It is another object of this invention that the bodily fluids can be blood, serum, lymph, urine, sweat, mucus, sputum, saliva, semen, spinal fluid, interstitial fluid, synovial fluid, cerebrospinal fluid, gingival fluid, vaginal fluid, and pleural fluid. Furthermore, the tissue can be liver, brain, muscle, adipose tissue, and kidney.

It is an object of this invention to have a kit useful in diagnosing damage or disease in tissue containing ALT2. This kit has a measurer of ALT2 levels in a sample of bodily fluids and an indicator for determining if amount of ALT2 measured by the ALT2 measurer falls in a range associated with damage or a specific disease in the ALT2 containing tissue. It is further object of this invention that the kit may also contain a measurer of ALT1 levels in a sample of bodily fluids and an indicator for determining if amount of ALT1 measured by the ALT1 measurer falls in a range associated with damage or a specific disease in the ALT2 containing tissue. The ALT2 measurer and the ALT1 measurer can be a biologic assay, an antibody-based assay, an enzyme linked immunosorbent assay, a Western blot, a rapid immunoassay, and a radioimmunoassay.

It is another object of this invention to have a diagnostic kit useful for diagnosing damage or disease to ALT2 containing tissue and/or ALT1 containing tissue. This diagnostic kit can contain ALT2 specific antibodies (polyclonal or monoclonal), immunoassay reagents, and a positive and negative control. This kit can also have ALT1 specific antibodies (polyclonal or monoclonal). This kit can have a means for determining if a measurement of ALT2 and/or ALT1 indicates a diagnosis of damage or disease in ALT2 containing tissue and/or ALT1 containing tissue. The kit can also have instructions indicating when a level of ALT1 and/or ALT2 is indicative for diagnosis of damage or disease in tissue containing ALT2 or ALT1.

It is an object of this invention to have a kit useful in determining when there are altered levels of ALT2 in bodily fluids (altered can be higher than normal or lower than normal). This kit can have a measurer of ALT2 levels in a bodily fluids sample and an indicator for determining if the ALT2 level measured falls in a range associated with a specific condition. It is a further object of this invention that the kit can determine when there are altered levels of ALT1 in bodily fluids (altered can be higher than normal or lower than normal). This kit can also have a measurer of ALT1 levels in a bodily fluids sample and another indicator for determining if the ALT1 level measured falls in a range associated with a specific condition. Furthermore, this kit can have a third indicator for comparing the values of ALT 1 and ALT2 and determining if the levels of ALT1 and ALT2 fall in a range associated with a specific condition. The measurer of this kit can be selected from one or more of the following: a biologic assay, an antibody-based assay, an enzyme linked immunosorbent assay, a Western blot, a rapid immunoassay, and a radioimmunoassay.

It is an object of this invention to have a method for producing ALT2. The ALT2 produced can be the same as the amino acid sequence of SEQ ID NO: 2 or a homolog, fragment, or variant. This method involves cloning the DNA encoding for ALT2 in an expression vector, introducing the expression vector into a host cell to produce a recombinant host cell, and subjecting to the recombinant host cell to conditions such that ALT2 is expressed. It is a further object of this invention that the ALT2 expressed can be isolated and purified. The DNA sequence placed in the plasmid can be the sequence of SEQ ID NO: 1 or any sequence which encodes for a variant, homolog, or fragment of ALT2.

It is another object of this invention to have a method for diagnosing a condition associated by altered levels of ALT2 and/or ALT1 in bodily fluids in an animal, including but not limited to human and mammal. This method involves contacting a sample of bodily fluids with at least one antibody which specifically binds to ALT2, detecting the ALT2 antibody which is bound to ALT2, and comparing the amount of detected ALT2 antibody to a known quantity for an animal (including but not limited to human and mammal) without the condition. In this method when the quantity of detected ALT2 antibody differs sufficiently from the known quantity from an animal without the condition, then it indicates that the animal has the condition. In addition, the method also can involve contacting the sample of bodily fluids with at least one antibody which specifically binds to ALT 1, detecting the ALT1 antibody which is bound to ALT1, and comparing said amount of detected ALT1 antibody to a known quantity for an animal without the condition. In this method when the quantity of detected ALT1 antibody differs sufficiently from the known quantity from an animal without the condition, then it indicates, that the animal has the condition. Furthermore, this method can also involving comparing the amount of ALT2 antibody detected to the total amount of antibody detected and/or to the amount of ALT1 antibody detect; and/or the amount of ALT1 antibody detected to the total amount of antibody detected and/or to the amount of ALT2 antibody detected. Again, the condition is indicated if the amount of ALT2 antibody detected when compared to the amount of ALT 1 antibody detected or the total amount of antibody detected falls within a certain range. Again, the condition is indicated if the amount of ALT1 antibody detected when compared to the amount of ALT2 antibody detected or the total amount of antibody detected falls within a certain range. It is a further object of this invention that the bodily fluids for this method can be selected from the following group: blood, serum, lymph, urine, sweat, mucus, sputum, saliva, semen, spinal fluid, interstitial fluid, synovial fluid, cerebrospinal fluid, gingival fluid, vaginal fluid, and pleural fluid.

### BRIEF DESCRIPTION OF THE FIGURES

FIG. 1 shows the cDNA sequence of ALT2 (SEQ ID NO: 1).

FIG. 2 shows the deduced amino acid sequence of ALT2 (SEQ ID NO: 2) compared with previously published human ALT (ALT1) amino acid sequence (SEQ ID NO: 3).

### DETAILED DESCRIPTION OF THE PREFERRED EMBODIMENTS

The present invention covers the nucleotide and amino acid sequences of ALT2, antibodies specific to ALT2, antibodies specific to ALT1, and the use of these polypeptides, polynucleotides, and antibodies to diagnose various diseases and conditions in tissue that produce ALT2, such as fatty liver and Syndrome X, and to differentially diagnose liver injury caused by fatty liver (liver steatosis) and by alcohol, trauma, infection, toxicity, and other causes of liver damage.

This invention also includes homologs and functional fragments of ALT2 as well as expression vectors containing ALT2 polynucleotide sequences and recombinant host cells which contain an expression vector containing ALT2 polynucleotide sequences.

For this application, homology is often measured using sequence analysis software (e.g., Sequence Analysis Software Package of the Genetics Computer Group, University of Wisconsin Biotechnology Center, 1710 University Avenue, Madison, Wis. 53705 or the NCBI BLAST program). Such software matches similar sequences by assigning degrees of homology to various substitutions, deletions, substitutions, and other modifications.

The term “functional fragments” include those fragments of SEQ ID NO: 2 or other proteins that have a similar amino acid sequence as that of the ALT2 polypeptide, that retains the function, activity, or immunobiological properties of ALT2. One of skill in the art can screen for the functionality of a fragment by using the examples provided herein, where full-length ALT2 is described. It is also envisioned that fragments ALT2 can be identified in a similar manner.

By “substantially identical” is also meant an amino acid sequence which differs only by conservative amino acid substitutions, for example, substitution of one amino acid for another of the same class (e.g., valine for glycine, arginine for lysine, etc.) or by one or more non-conservative substitutions, deletions, or insertions located at positions of the amino acid sequence which do not destroy the function of the protein assayed, (e.g., as described herein). Preferably, such a sequence is at least 85%, and more preferably from 90%, 91%, 92%, 93%, 94%, 95%, 96%, 97%, 98%, 99%, to 100% homologous at the amino acid level to SEQ ID NO: 2.

By a “substantially pure polypeptide” is meant an ALT2 polypeptide that has been separated from components that naturally accompany it. Typically, the polypeptide is substantially pure when it is at least 60%, by weight, free from the proteins and other naturally occurring molecules with which it is typically associated. Preferably, the preparation is at least 75%, 80%, 90%, 95%, and most preferably at least 99%, by weight, ALT2. A substantially pure ALT2 polypeptide can be obtained, for example, by extraction from a natural source; by expression of a recombinant nucleic acid encoding ALT2 polypeptide; or by chemically synthesizing the protein. Purity can be measured by any appropriate method, e.g., column chromatography, polyacrylamide gel electrophoresis, or by HPLC analysis.

A protein is substantially free of naturally associated components when it is separated from those contaminants that accompany it in its natural state. Thus, a protein that is chemically synthesized or produced in a cellular system different from the cell from which it naturally originates will be substantially free from its naturally associated components. Accordingly, substantially pure polypeptides include those derived from eukaryotic organisms but synthesized in E. coli or other prokaryotes.

As would be evident to one skilled in the art, the polynucleotide molecules of the present disclosure can be expressed in a variety of prokaryotic and eukaryotic cells using regulatory sequences, vectors, and methods well established in the literature.

ALT2 polypeptide produced according to the present description can be purified using a number of established methods such as affinity chromatography using an anti-ALT2 antibodies coupled to a solid support. Fusion proteins of an antigenic tag and ALT2 can be purified using antibodies to the tag. Optionally, additional purification is achieved using conventional purification means such as liquid chromatography, gradient centrifugation, and gel electrophoresis, among others. Methods of protein purification are known in the art and can be applied to the purification of recombinant ALT2 polypeptide described herein. Purification of ALT2 polypeptide is discussed more completely below.

Construction of ALT2 encoded fusion proteins is also contemplated. Fusion proteins will typically contain additions, substitutions, or replacements of one or more contiguous amino acids of the native ALT2 polypeptide with amino acid(s) from a suitable fusion protein partner. Such fusion proteins are obtained using recombinant DNA techniques well known by one of skill in the art. Briefly, DNA molecules encoding the hybrid ALT2 protein of interest are prepared using generally available methods such as PCR mutagenesis, site-directed mutagenesis, and/or restriction digestion and ligation. The hybrid DNA is then inserted into expression vectors and introduced into suitable host cells.

Recombinant gene expression vectors comprising ALT2, or portions thereof, can be constructed in a variety of forms well-known in the art. Preferred expression vectors include plasmids and cosmids. Expression vectors include one or more fragments of ALT2. Typically, an expression vector will comprise ALT2 polynucleotide sequence (SEQ ID NO: 1).

As used herein, the phrase “operatively encode” refers to one or more protein coding regions associated with those regulatory sequences required for expression of the polypeptide encoded by the coding region. Examples of such regulatory regions including promoter binding sites, enhancer elements, ribosome binding sites, and the like. Those of ordinary skill in the art will be able to select regulatory sequences and incorporate them into the recombinant expression vectors described herein without undue experimentation. For example, suitable regulatory sequences for use in various eukaryotic and prokaryotic systems are described in Ausubel, et al., Short Protocols in Molecular Biology, 3^{rd} ed., John Wiley & Sons, Inc, New York, 1997, which is hereby incorporated by reference in its entirety.

Expression vectors for use with ALT2 will typically contain regulatory sequences derived from a compatible species for expression in the desired host cell. For example, when E. coli is the host cell, the host cell population can be typically transformed using pBR322, a plasmid derived from an E. coli species. (Bolivar, et al., Gene, 2:95, 1977). pBR322 contains genes for ampicillin (AMPR) and tetracycline resistance and thus provides easy means for identifying transformed cells.

Promoters suitable for use with prokaryotic hosts illustratively include the beta-lactamase and lactose promoter systems (Chang, et al., Nature, 275:615, 1978, and Goeddel, et al., Nature, 281:544, 1979), alkaline phosphatase, the tryptophan (trp) promoter system (Goeddel, Nucleic Acids Res., 8:4057, 1980) and hybrid promoters such as the taq promoter (de Boer, et al., Proc. Natl. Acad. Sci. USA, 80:21-25, 1983). Other functional bacterial promoters are also suitable. Their nucleotide sequences are generally known in the art, thereby enabling a skilled worker to ligate them to a polynucleotide which encodes the peptide of interest (Siebenlist, et al., Cell, 20:269, 1980) using linkers or adapters to supply any required restriction sites.

In addition to prokaryotes, eukaryotic microbes such as yeast cultures can also be used as source for the regulatory sequences. Saccharomyces cerevisiae is a commonly used eukaryotic host microorganism. Suitable promoting sequences for use with yeast hosts include the promoters for 3-phosphoglycerate kinase (Hitzeman, et al., J. Biol. Chem., 255:2073, 1980) or other glycolytic enzymes (Hess, et al. J. Adv. Enzyme Reg. 7:149, 1968; and Holland, Biochemistry, 17:4900, 1978) such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degraded enzymes associated with nitrogen metabolism, metallothionine, glyceraldehyde-3-phosphate dehydrogenase, and enzymes responsible for maltose and galactose utilization. Yeast enhancers also are advantageously used with yeast promoters.

In another embodiment, a recombinant virus is used as the expression vector. Exemplary viruses include the adenoviruses, adeno-associated viruses, herpes viruses, vaccinia, or an RNA virus such as a retrovirus or an alphavirus. Preferably, the retroviral vector is a derivative of a murine or avian retrovirus. Preferably the alphavirus vector is derived from Sindbis or Semliki Forest Virus. AU of these vectors can transfer or incorporate a gene for a selectable marker so that transduced cells can be identified and generated.

By inserting one or more sequences of interest into the viral vector, along with another gene which encodes the ligand for a receptor on a specific target cell, for example, the vector is now target specific. Retroviral vectors can be made target specific by inserting, for example, a polynucleotide encoding a sugar, a glycolipid, or a protein. Preferred targeting is accomplished by using an antibody to target the retroviral vector, such as to the vicinity of a mucosal inductor site, using a MALT-specific antibody. Those of skill in the art will know of, or can readily ascertain without undue experimentation, specific polynucleotide sequences which can be inserted into the retroviral genome to allow target specific delivery of the retroviral vector containing the polynucleotides of interest.

Construction of suitable vectors containing desired coding, non-coding and control sequences employ standard ligation techniques. Isolated plasmids or DNA fragments are cleaved, tailored, and re-ligated in the form desired to construct the plasmids required.

For example, for analysis to confirm correct sequences in plasmids constructed, the ligation mixtures can be used to transform a host cell and successful transformants selected by antibiotic resistance where appropriate. Plasmids from the transformants are prepared, analyzed by restriction and/or sequenced by, for example, the method of Messing, et al., (Nucleic Acids Res., 9:309, 1981), the method of Maxam, et al., (Methods in Enzymology, 65:499, 1980), or other suitable methods which will be known to those skilled in the art. Size separation of cleaved fragments is performed using conventional gel electrophoresis as described, for example, by Maniatis, et al., (Molecular Cloning, pp. 133-134, 1982).

Host cells can be transformed with the expression vectors described herein and cultured in conventional nutrient media modified as is appropriate for inducing promoters, selecting transformants or amplifying genes. The culture conditions, such as temperature, pH and the like, are those previously used with the host cell selected for expression, and will be apparent to the ordinarily skilled artisan.

Cloning of ALT2

To clone ALT2, a human adipose cDNA library (BD Bioscience Clontech, Palo Alto, Calif.) is used to generate expressed sequence tags (ESTs) by PCR amplification and sequencing according to BD Bioscience Clontech's protocol. The primers for amplifying and sequencing of the ESTs are derived from the TripEX vector, which is used for constructing the human adipose cDNA library. Primer P922 (5′-AATACGACTCACTATA GGGCGAATTGG-3′ SEQ ID NO: 4 ) and primer P923 (5′-CTCGGGAAGCGCGCCATTGTGTTGGT-3′ SEQ ID NO: 5) are used for PCR and primer P927 (5′-GTTGGTACCCGGGAATTC-3′ SEQ ID NO: 6) is used for sequencing. The PCR conditions were 94° C. for 2 minutes followed by 10 cycles of 94° C. for 15 seconds, 58° C. for 30 seconds and 68° C. for 4 minutes, 20 cycles of 94° C. for 15 seconds, 58° C. for 30 seconds and 68° C. for 25 seconds plus 5 seconds/cycle and a final extension at 72° C. for 7 minutes. The 5′-end of human ALT2 is determined by 5′-rapid amplification of cDNA end with an adaptor primer (AP1) and ALT2-specific primer p1106 5′-TAGGTGCATAGTGCCATCAC-3′ [nucleotides (nt) 447-428 in A Y029173)] (SEQ ID NO: 7) using the human adipose Marathon cDNA kit (BD Bioscience Clontech). PCR used 30 cycles of 94° C. for 30 seconds, 56° C. for 30 seconds, and 72° C. for 1 minute.

The resultant PCR product is directly sequenced using BigDye sequencing chemistry (Applied Biosystems, Norwalk, Conn.). Sequencing reaction is performed in20 μl containing 100 ng of resultant PCR product, 5 pmol of primer, 4 μl of BigDye sequencing reaction mixture. Cycling conditions are 96° C. for 15 seconds, 50° C. for 10 seconds and 60° C. for 4 minutes for 25 cycles. The reaction mixture is purified by passing a Sephdex-G50 column and is dried in a vacuum centrifuge. Electrophoresis and data analysis of samples is performed using ABI PRISM 377 Sequencer. Thus, the full-length sequence is obtained, confirming that this cDNA encodes a novel ALT homologue that has been named ALT2. The 3,957 bp cDNA contains a 1,569 bp open reading frame that encodes 459 amino acid residues. Two potential in-frame ATG initiation codons are found 51 and 24 amino acids apart in ALT2 and ALT1 protein sequences, respectively. No Kozak sequences are observed in either of these ATGs in the ALT2 sequence and therefore, which ATG is likely to be more efficiently utilized for translation initiation or whether the longer amino terminus of ALT2 may contain a targeting signal remains to be determined. Significant conservation is observed from the second ATG, suggesting that the protein encoded after this ATG is likely to be functionally more important.

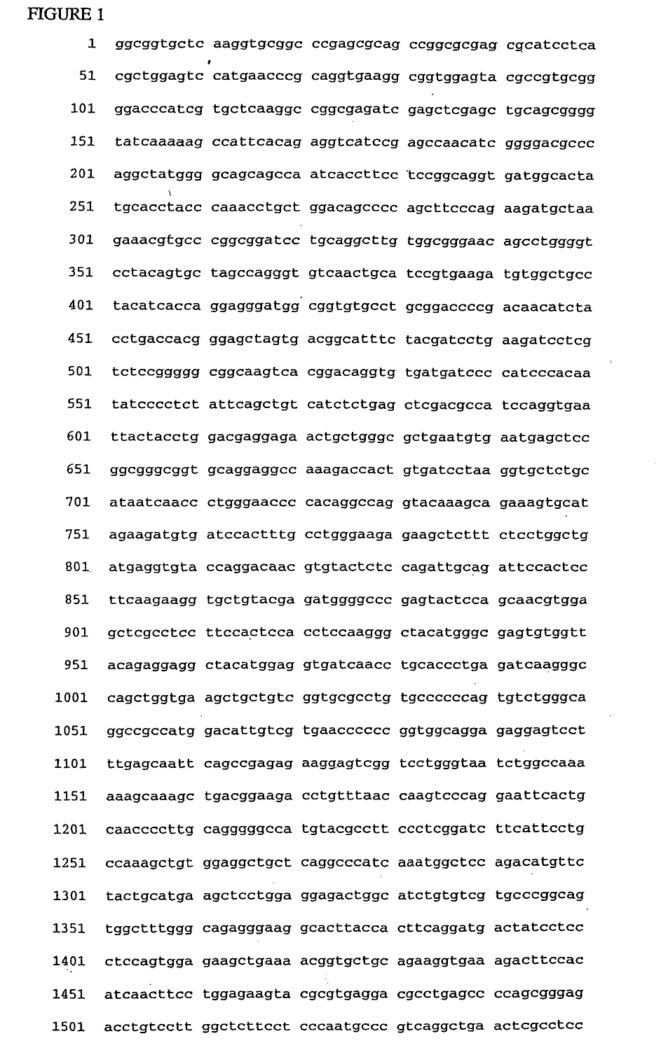

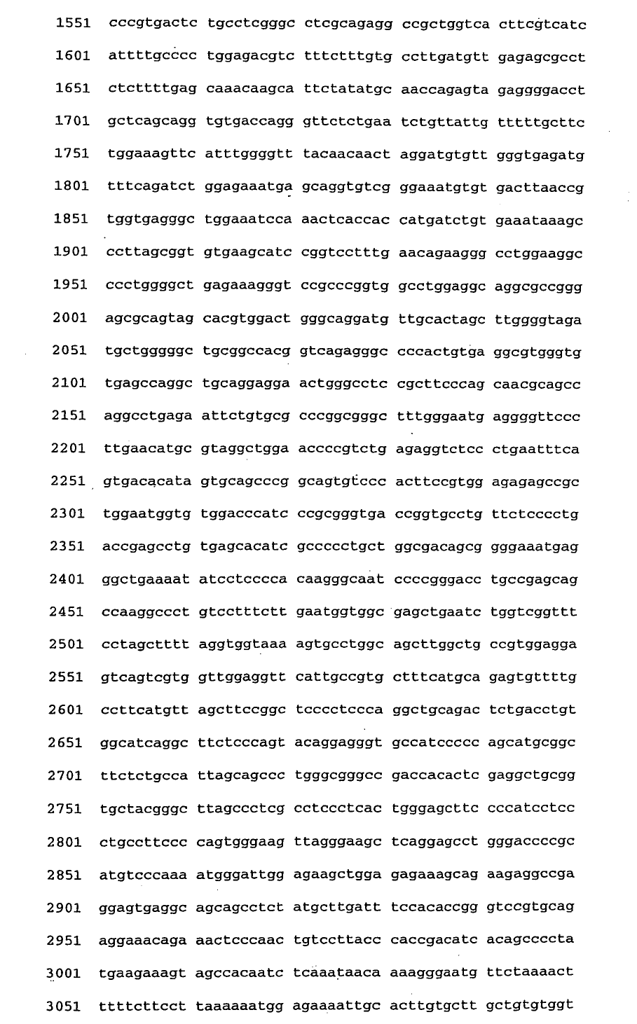

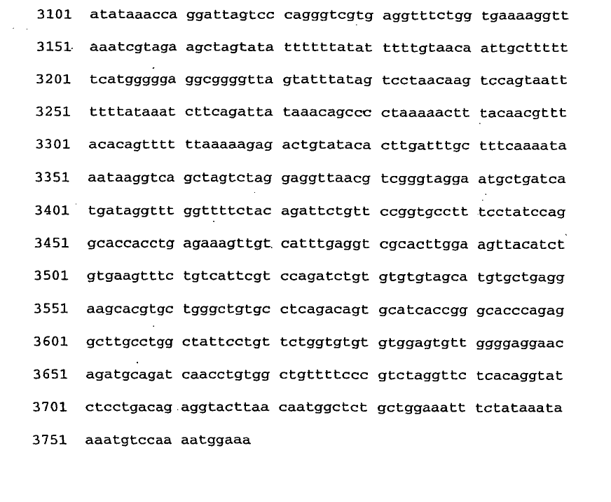

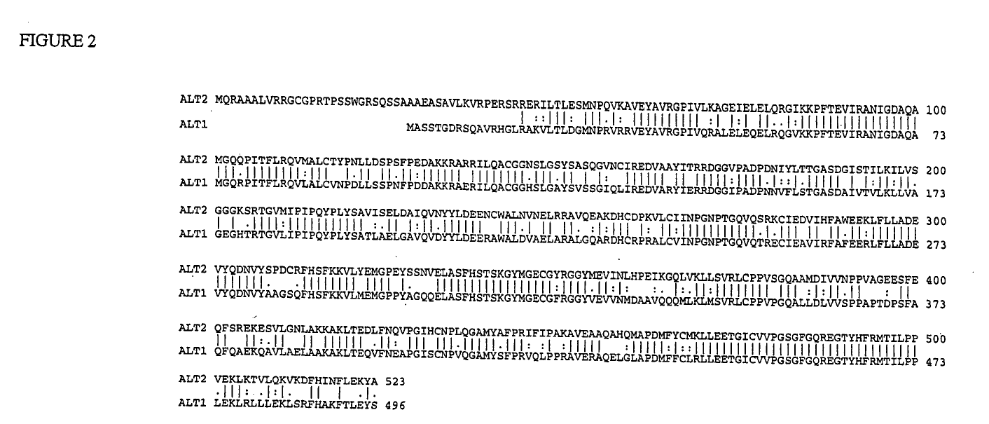

FIG. 1 contains the cDNA sequence (SEQ ID NO: 1) of ALT2. The nucleotide and amino acid sequences of ALT2 were deposited with GenBank (accession number AY029173) on Apr. 16, 2001 but publication was withheld until Mar. 1, 2002. The deduced 459 amino acid sequence of human ALT2 is 69% identical and 78% similar to human ALT1 amino acid sequence (SEQ ID NO: 3), as determined by using Program Bestfit from GCG10 (FIG. 2). Note that ALT2 is 27 amino acids longer at the amino terminus than ALT1. The calculated molecular weights of ALT1 and ALT1 are 59 kDa and 55 kDa; the isoelectric points are 6.42 and 7.11, respectively.

Conserved domain analysis (http://www.ncbi.nlm.nih.gov/Structure/cdd/wrpsb.cgi) reveals that ALT2 contains the complete domain of transaminase class I (aminotrans_{—}2, pfam00155), which includes aspartate transaminase and tyrosine transamiinase, and a partial domain of transaminase class II (aminotrans_{—}2, pfam00222). These structures are conserved among various transaminases including aspartate, tyrosine and histininol-phosphate aminotransferases.

A search using ALT2 cDNA as a probe against the High Throughput Genomic Sequences (“HTGS”) database from GenBank reveals an almost perfect match to three unfinished genomic sequences (accession numbers AC007225, AC018845, and AC007338) which consists of overlapping human genomic bacterial artificial clones (BACs) from chromosome 16. The BACs containing the ALT2 gene were provided by Los Alamos Laboratory, New Mexico. The genomic structure is determined by a combination of alignment of ALT2 cDNA to the genomic sequences with reference to the GenBank sequence and partial PCR amplification and sequencing of the region where the order and sequence in the clones are ambiguous. The genomic sequences that cover exons from 1 to 4 and from 5 to 12 are contained in single contigs, which are further verified by PCR amnplification. The gap between exon 4 and exon 5 is closed by PCR amplification of the BAC clone BP11-169E6 using primers from exon 4 and 5 (Primer P1145: 5′-CAGGTGATGGCACTATGCACCT-3′ [(nt) 425-446 in AY029173] (SEQ ID NO: 8) and primer P1090: 5′-CTCCCGTCCTCAGGTAGATGTTGT-3′ [(nt) 653-630 in AY029173] (SEQ ID NO: 9)). The PCR reaction conditions are as described above. The resulting PCR product is confirmed by sequencing analysis. After assembly, the human ALT2 gene consists of 12 exons and span approximately 50 kb in the genome. The splicing sites and exon-intron boundaries are presented in Table 1. The genomic organization is completely conserved between ALT2 and ALT1, except that the ALT1 gene is much shorter, only about 3 kb.

TABLE 1

| | | | | |
|---|---|---|---|---|
| Exon | Exon size | | Intron size | |
| No. | (bp) | 5′-splice donor | (kb) | 3′-splice acceptor |

| | | | | |
|---|---|---|---|---|
| 1 | ˜72 | CGACAGgcacgt | 0.2 | tgccagGGTTTC |
| | | (SEQ ID NO: 10) | | (SEQ ID NO: 11) |
| 2 | 265 | CAGCGGgtgagc | 12.7 | gcccagGGTATC |
| | | (SEQ ID NO: 12) | | (SEQ ID NO: 13) |
| 3 | 90 | CGGCAGgtgagc | 2.9 | ccccagGTGATG |
| | | (SEQ ID NO: 14) | | (SEQ ID NO: 15) |
| 4 | 109 | GCCTGGgtgagg | 6.1 | ttacagGGTCCT |
| | | (SEQ ID NO: 16) | | (SEQ ID NO: 17) |
| 5 | 134 | ATTTCTgtacgt | 2.7 | ttgcagACGATC |
| | | (SEQ ID NO: 18) | | (SEQ ID NO: 19) |
| 6 | 244 | CCACAGgtctgc | 6.7 | ttatagGCCAGG |
| | | (SEQ ID NO: 20) | | (SEQ ID NO: 21) |
| 7 | 80 | GATGAGgtaaga | 1.9 | ccgcagGTGTAC |
| | | (SEQ ID NO: 22) | | (SEQ ID NO: 23) |
| 8 | 137 | GGGCGAgtacgt | 3.5 | ctccagGTGTGG |
| | | (SEQ ID NO: 24) | | (SEQ ID NO: 25) |
| 9 | 175 | AGCCGAgtgagt | 2.0 | catcagGAGAAG |
| | | (SEQ ID NO: 26) | | (SEQ ID NO: 27) |
| 10 | 156 | GCTCAGgtctgg | 2.4 | ccatagGCCCAT |
| | | (SEQ ID NO: 28) | | (SEQ ID NO: 29) |
| 11 | 113 | CTTCAGgtatga | 1.9 | tgccagGATGAC |
| | | (SEQ ID NO: 30) | | (SEQ ID NO: 31) |
| 12 | 2382 | | | |

Because HTGS data from GenBank are not final and may contain errors, chromosomal localization of ALT2 is determined by screening of a human/hamster radiation hybrid array. The Stanford GeneBridge 3 radiation hybrid panel (Research Genetics, Invitrogen, Carlsbad, Calif.), is utilized for mapping of the chromosomal localization with the primers 5′-GGCAGGATGTTGCACTAGCTF-3′ (SEQ ID NO: 32) and 5′-ACTATGTGTCACTGA-3′ (SEQ ID NO: 33) in the 3′-untranslated region of ALT2 (Gong, et al., Uncoupling protein-3 is a mediator of thermogenesis regulated by thyroid hormone, beta3-adrenergic agonists, and leptin, J. Biol. Chem., 272:24129-24132 (1997)). PCR used 30 cycles of 94° C. for 30 seconds; 56° C. for 30 seconds; and 72° C. for 30 seconds. Data analysis is performed using the Stanford University's web site (http://www-shgc.stanford.edu/RH/) (Stanford, Calif.).

The resulting code, 00000010000000010000010000000000011000010010000010001000000200111000000100 00100000, is assigned to chromosome 16 (26 cRs) with a lod score of 8.5. This result is consistent with a previous mapping of ALT activity to chromosome 16, which used hybrids of human leukocytes and Chinese hamster fibroblasts, and is distinct from ALT1, which is located on chromosome 8.

Northern analysis is performed to determine the tissue distribution of ALT2 and ALT1. Adipose tissue total RNA is prepared with Trizol (Life Technologies Inc., Bethesda, Md.) from human and rhesus monkey fat tissues (Gong, et al., Genomic structure and promoter analysis of the human obese gene, J. Biol. Chem., 271:3971-3974 (1996); Hotta, et al., Circulating concentrations of the adipocyte protein adiponectin are decreased in parallel with reduced insulin sensitivity during the progression to type 2 diabetes in rhesus monkeys, Diabetes, 50:1126-1133 (2001)). In brief, adipose tissues are homogenized in Trizol (1 ml of Trizol per 100 mg of tissue with Polytron). The resulting homogenate is briefly centrifuged (12,000×g, 10 minutes) and the upper oil/triglyceride phase is removed before adding 0.2 μl chloroform /1 ml Trizol. The mixture is then votexed and centrifuged, and the total RNA contained in the aqueous phase is transferred into a fresh tube and precipitated with isopropanol. All other RNAs are purchased from BD Bioscience Clontech. Fifteen micrograms of total RNA per lane are loaded onto a polyacrilimide gel for Northern analysis. The human ALT2 probe corresponds to bases 615 to 3957 in GenBank AY029173, and the human ALT1 probe is a 2.1 kb insert of the sequence-confirmed IMAGE clone 2129833 (Research Genetics, Invitrogen). Probes are random-labeled (Stratagene, La Jolla, Calif.) with ³²P-dCTP, hybridization is carried out at 65° C. in Rapid-hyb buffer (Amersham Pharmacia Biotech, Inc., Piscataway, N.J.), and blots are washed twice with 0.5×SSC/1% SDS at 65° C. (stringent wash).

The ˜3.9 kb human ALT2 mRNA is expressed at high levels in muscle, kidney, brain and fat, and less in liver and breast. In contrast, the 1.4 kb human ALT1 mRNA is moderately expressed in kidney, liver, fat and heart. High level of expression of ALT2, but not ALT1, is also detected in monkey adipose tissue. The sizes of ALT2 and ALT1 transcripts agree with the length of the predicted and reported cDNAs. Only one band is detected for each ALT transcript, indicating that there is no obvious alternate splicing in these two genes. ALT2 appears to be the predominant ALT and is more ubiquitously expressed than ALT1 at the mRNA level. This finding is consistent with the abundance of ALT transcripts in GenBank where there were about 228 ALT2 ESTs, but only 11 ALT1 ESTs in a total of 2.5 million ESTs (GenBank Builder #130).

Bacterial Expression of ALT1 and ALT2

The coding region of ALT2 is amplified by PCR (28 of cycles of 94° C. for 30 seconds, 56° C. for 30 seconds, and 72° C. for 1.5 minutes with final extension of 7 minutes at 72° C., using the High Fidelity PCR System (Roche Diagnostics, Hoffman-LaRoche, Basal, Switzerland)) with an NdeI-linked primer p1266 5′-acctgaattcatATGCAGCGGGCGGCGGCGCT-3′ (nt 95 to 114, AY029173) (SEQ ID NO: 34) and a HindIII-linked primer p1117 5′-ggctcagaagcttTCACGCGTACTTCTCCAGCAA-3′ (nt 1666 to 1646, AY029173) (SEQ ID NO: 35) using human muscle first-strand cDNA (Clontech). The resulting PCR product is digested with Ndel/HindIII and subcloned into pPET28a (Novagen Inc., Madison, Wis.) upstream of the polyhistadine tag, creating plasmid pPET28-ALT2. The absence of mutations in the inserted ALT2 cDNA is verified by DNA sequence analysis.

The same approach is used to clone the coding region of ALT1 into pPET28a with primer p1118 5′-ctgggtagacatATGGCCTCGAGCACAGGTGAC-3′ (nt 268 to 288, NM_{—}005309) (SEQ ID NO: 36) and p1119 5′-ccccagctgaagcttTCAGGAGTACTCGAGGGTGAAC-3′ (nt 1158 to 1137, NM_{—}005309) (SEQ ID NO: 37) with ALT1 plasmid (IMAGE clone 2129833) as template, creating plasmid pPET28-ALT1.

To express ALT2 and ALT1 proteins, competent E. coli. (Novagen, Inc.) are transform with pPET28-ALT2, pPET28-ALT1, or pPET28a (negative control). A fresh colony of the transformants is grown in 50 ml LB media containing 30 μg/ml kanamycin to an OD₆₀₀ of 0.7, at which time isopropyl-β-D-thiogalactopyranoside (IPTG) is added (1 mM, final concentration) to induce expression of the recombinant proteins. Cell pellets are harvested from 20 ml cultures before and after 3 hours of induction, and are resuspended in 5 ml of 1× PBS buffer, followed by a brief sonication (3 times for 10 seconds, setting 3, Fisher 550 Sonic Dismembrator). Cell lysates are centrifuged at 10,000 g for 15 minutes at 4° C. The supernatants are collected for standard SDS-PAGE analysis and enzyme activity.

The GPT Optimized Alanine Aminotransferase kit (Sigma Diagnostics, St. Louis, Mo.) is used to measure ALT activities. Briefly, 0.5 ml of cell lysate is incubated with a 2.5 ml mixture of reagent A and B containing L-alanine, NADH, LDH and 2-oxoglutarate at 25° C. Absorbance at 340 nm is recorded at 1, 2 and 3 minutes after incubation. The slope of absorbance decrease is proportional to ALT activity. Protein concentration of cell lysate is determined by Coomassie Brilliant Blue G250 (BioRad Laboratories, Hercules, Calif.) using bovine serum albumin as standard. Final ALT activities are corrected by protein concentration of cell lysate. One unit of ALT activity is defined as the amount of enzyme which catalyzes the formation of 1 μmol/L of NAD per minute under conditions of the assay at 25° C.

Under basal conditions, significant ALT activity is observed in cell lysates from E. coli transformed with pPET28-ALT2 (121 units/mg protein) and pPET28-ALT1 (86 units/mg protein), with much less activity in cell lysate from bacteria transformed with pPET28a (16 units/mg protein). The higher basal activity in the ALT-containing transformants resulted from “leaky” expression of the recombinant proteins. IPTG induced ALT2 activity by 2.8 fold and ALT1 activity by 2 fold but does not induce ALT activity in the negative control bacteria.

For SDS-PAGE analysis, the supernant of the IPTG induced E. coli transformed with pPET28-ALT1 or pPET28-ALT2 or pPET28a is collected as described above. The supernant is then run on a 10% SDS-PAGE gel. The gel is either stained with Coomassie Blue or is immuno-blotted with anti-histidine antibody. Protein bands are visible at approximately 62 kDa and 58 kDa after IPTG induction, corresponding to ALT2 (calculated molecular weight of 58 kDa) and ALT1 (calculated molecular weight of 54 kDa). These data confirm that ALT2 cDNA encodes a functional alanine transaminase.

ALT2 Expression in Fatty Liver

Because ALT2 is highly expressed in adipose tissue, the expression of ALT2 in fatty liver in the obese state is examined. Obese mice (ob/ob) are well-recognized models for nonalcoholic fatty liver disease (NAFLD), a type of liver disease that is strongly associated with obesity and type 2 diabetes (Koteish and Diehl, Animal models of steatosis, Semin. Liver. Dis., 21(1):89-104 (2001)). 4-month-old obese mice(ob/ob) or lean mice (ob/+) (Jackson Laboratory, Bar Harbor, Me.) are sacrificed and the liver is isolated. Total RNA is prepared with Trizol from the isolated livers as described above. Fifteen micrograms of total RNA per lane are loaded onto an polyacrylamide gel for Northern analysis. The mouse ALT1 cDNA (amino acids 106-294, (EST, IMAGE clone 521920, AA106294) Research Genetics), random-labeled (Stratagene) with ³²P-dCTP, is used as probe. Hybridization is carried out at 65° C. in Rapid-hyb buffer (Amersham), and blots are washed twice with 0.5×SSC/1% SDS at 65° C. (stringent wash). RNA levels are quantitated by PhosphorImager and statistical significance is determined using Student's t-test. Hepatic ALT2 expression increases 1.9-fold in obese (ob/ob) compared to lean (ob/+) mice (p<0.05). By contrast, ALT1 expression does not differ between obese and non-obese mice. Thus, ALT2 expression is specifically elevated in fatty liver.

ALT2 As An Indicator of Disease or Injury of ALT2 Producing Tissue

As indicated above, the current assay for ALT does not distinguish between ALT1 and ALT2. As such, the assay has severe limitations in its accuracy of liver damage. The liver contains both ALT1 and ALT2, and ALT2 mRNA levels are elevated in animal models of fatty liver disease while ALT1 mRNA levels do not change. Assays that differentiate between ALT1 and ALT2 can be useful in the differential diagnosis of liver injuries in that one can distinguish between liver damage caused by steatosis and damage caused by alcohol, drug and chemical toxicity, trauma, infection, and other types of injury.

Furthermore, adipose tissue contain higher levels of ALT2 than ALT1. An assay for ALT2 or for both ALT1 and ALT2 may be useful for the diagnosis of Syndrome X and other disorders of the adipose tissue.

Finally, having an assay which distinguishes between ALT1 and ALT2 in blood or in tissue may be useful in diagnosis of injury or disease in tissue which produce or contain ALT2.

To diagnose liver injury or disease in an animal (human, mammal, or other), one can determine the total amount of ALT polypeptides in the blood or other bodily fluids by using antibodies to ALT polypeptides (as described below). A total ALT level of 40 IU/L or great is indicative of liver injury or disease. Alternatively, a total amount of ALT polypeptides in blood or other bodily fluids is 1.5-2 fold greater than the upper limit of total ALT polypeptide levels in blood or other bodily fluids in an animal without injury or disease is indicative of liver injury or disease.

Then to determine the cause of the liver damage, one should examine the relative amount of ALT2 and ALT1 polypeptides in the blood or other bodily fluids by using ALT1-specific antibodies and ALT2-specific antibodies (as described below). The amount of ALT2 and ALT1 polypeptides can also be determined using enzymatic assays or other well-known in the art-field assays which distinguish between ALT1 and ALT2 activity. When the amount of ALT2 polypeptide in blood or other bodily fluids is more than 1.5 times higher than the amount of ALT1 polypeptide in blood or other bodily fluids, then the liver damage is usually caused by liver steatosis. When the amount of ALT2 polypeptide in blood or other bodily fluids is less than 1.5 times higher than the amount of ALT1 polypeptide in blood or other bodily fluids, then the liver damage is usually caused by an infection, toxicity, or trauma.

For diseases and conditions of tissue that produce ALT2 (other than the liver), the approach is similar. One can diagnose injury or disease in tissue that produce ALT2 when the amount of ALT2 polypeptide present in the blood or other bodily fluids of the animal is 1.5-2 fold greater than the upper limit of the level of ALT2 polypeptide in the blood or other bodily fluids of an animal without injury or disease, as specified by the laboratory performing the assay. ALT2-specific antibodies can be used to determine the amount of ALT2 in blood or other bodily fluids (as described below).

In a blood or other bodily fluids assay, if the amount of ALT2 polypeptide is greater than 0.5 times higher than the amount of ALT1 polypeptide, and the animal has elevated levels of LDL-triglycerides, hypertension, and is over-weight, then it is likely that the animal has Syndrome X.

Similarly, if one uses tissue samples for diagnosis, then if the amount of ALT2 mRNA is greater than 1.5 times higher than the amount of ALT1 mRNA then the liver damage is usually caused by liver steatosis. Similarly if the amount of ALT2 mRNA in adipose tissue is 3 times higher the amount of ALT1 mRNA, this level is suggestive of a diagnosis for Syndrome X. One may need to also look at the levels of LDL-triglycerides, and test for hypertension and obesity to assist in the diagnosis for Syndrome X. For other tissue that produce ALT2, when the amount of ALT2 mRNA is 1.5-2 fold higher than the amount of ALT2 mRNA in an animal's tissue that is known not to have a disease or injury, then one can diagnose the animal with disease or injury for tissue that produce ALT2.

An assay for ALT1 and/or ALT2 can either be an assay of the amount of mRNA present in tissue or an assay of the amount of ALT1 and/or ALT2 protein in tissue or body fluids such as blood, serum, lymph, urine, sweat, mucus, sputum, saliva, semen, spinal fluid, interstitial fluid, synovial fluid, cerebrospinal fluid, gingival fluid, vaginal fluid, and pleural fluid. It may be more preferable to assay for the presence of the protein or fragments of the protein in fluids than to assay for the protein or fragments of the protein in tissue or mRNA in tissue.

mRNA Assay

An assay for mRNA for ALT1 and/or ALT2 requires the isolation of mRNA from tissue and/or fluid samples. A crude biological sample in a liquid medium such as serum, plasma, tissue sections or extracts, lymphocytes, tissue culture cells or the supernatant growth medium from tissue culture can be used in the assay without prior purification. Alternatively, a purified or partially purified sample can be assayed. Purification steps can include separation, extraction, cell disruption or homogenization of tissue or biological fluids. Additionally, carrier nucleic acids which do not react with a probe can be added to the sample during purification to reduce non-specific binding of nucleic acids.

Next, the sample can be treated with a detergent or a proteinase in order to solubilize and release the nucleic acids for assay. The addition of a proteinase is preferred in part because that treatment results in more stable and solubilized nucleic acids. Among the proteinases considered useful in this method are Proteinase K, trypsin, chymotrypsin, pronase, alkaline proteases, lysozyme and subtilisin. Another enzyme useful in isolating and purifying mRNA is DNAse. It is preferable to denature the mRNA isolated, using thermal denaturation, to disrupt the secondary structure of the mRNA.

Following disruption of the mRNA's secondary structure, the hybridization reaction with a labeled probe is performed under conditions suitable for selectively binding the labeled probe to the target mRNA. General methods for hybridization reactions and probe synthesis are disclosed in Molecular Cloning by T. Maniatis, E. F. Fritsch and J. Sambrook, Cold Spring Harbor Laboratory, 1982. The hybridization reaction can take place under a range of pH, salt and temperature conditions. The pH can vary from 6 to 9 with a preferred pH being 6.8 to 8.5. The salt concentration can vary from 0.15 M sodium to 0.9 M sodium. Other cations can be utilized as long as the ionic strength is equivalent to that specified for sodium. The temperature of the hybridization reaction can vary from 30° C. to 80° C. with a preferred temperature range between 45° C. and 70° C. Additionally, other compounds can be added to the hybridization reaction to promote specific hybridization at lower temperatures, such as at or approaching room temperature. Among the compounds contemplated for lowering the, temperature requirements is formamide.

It is noted that the labeled probe for assaying for the amount of mRNA of ALT2 in a sample is a polynucleotide that is complementary to and specific for ALT2 mRNA sequences. The length of the polynucleotide probe can range in size from 10 bases to over 4,000 bases, more preferably from 20 to 3,500 bases long. One preferred probe for ALT2 for a Northern blot analysis is approximately 3.3 kb long corresponding to nucleotides 615 to 3,957 in AY029173.

It is noted that the labeled probe for assaying for the amount of mRNA of ALT1 in a sample is a polynucleotide that is complementary to and specific for ALT1 mRNA sequences. The length of the polynucleotide probe can range in size from 10 bases to over 4,000 bases, more preferably from 20 to 3,500 bases long.

The usual methods of RNA detection are Northern blots, RNase protection, and reverse transcriptase-PCR (RT-PCR). These methods are well-known in the art field.

It is also noted that the polynucleotide probe is labeled with a substance which can be covalently attached to or firmly associated with a nucleic acid probe which will result in the ability to detect and quantitate the amount of probe. Among the substances contemplated are radioisotopes such as ³H, ¹²⁵I, ¹³¹I, ³²P, ³³P, ¹⁴C and ³⁵S; chemiluminescent molecules such as acridines or luminol; fluorescers such as fluorescein, phycobiliproteins, rare earth chelates, dansyl, rhodamine; enzyme substrates and inhibitors such as horseradish peroxidase, glucose oxidase, glucose-6-phosphate dehydrogenase, beta-galactosidase, pyruvate kinase, alkaline phosphatase, acetylcholinesterase; metal particles or other radiopaque molecules such as colloidal gold, magnetic particles; liposomes containing any of the above substances; antigens, such as proteins, carbohydrates or haptenic molecules and antibodies specific for those antigens; and glycolipids or glycoproteins which are members of specific binding pairs.

Following the hybridization reaction, the ALT2 mRNA hybridized to the labeled probe is separated from the unhybridized labeled probe to determine the quantity of hybridized probe and, thus, the quantity of ALT2 mRNA present in the sample. Gel exclusion or affinity chromatography can be used to separate the unhybridized probe from the hybridized probe. Similarly, for ALT1, following the hybridization reaction, the ALT1 mRNA hybridized to the labeled probe is separated from the unhybridized labeled probe to determine the quantity of hybridized probe and, thus, the quantity of ALT1 mRNA present in the sample. Again, gel exclusion or affinity chromatography can be used to separate the unhybridized probe from the hybridized probe. One preferred method of separation is accomplished by gel exclusion chromatography. The conditions for the separation method must be such that the hybridized target nucleic acid and detectable probe remain bound to each other. The separation solution may contain inhibitors of nucleases and, also, additional carrier nucleic acid sequences which do not interact with the probe.

Among the preferred methods of separating the hybridized probe from the unhybridized probe are gel exclusion chromatography through matrices composed of polyacrylamide, sepharose, cross-linked sepharose, agarose, cross-linked agarose or other similar materials. Products suitable for such gel exclusion chromatography include the Pharmacia Sephadex products designated G50, G100, and G200; and Pharmacia products designated Sepharose CL2B, 4B, 6B, S-200, S-400 and S-1000. Other suitable products from the BioRad Corporation include P-20, P-60, P-100, P-200, A-0.5 m and A-1.5 m.

Following the separation of hybridized probe from unhybridized probe, the presence of or quantity of hybridized probe is determined. The method of detection depends upon the type of label present on the probe. When the label is a radioisotope, the detection method can be by gamma or scintillation counting or by another method capable of detection, such as autoradiography or photographic detection. When the probe is labeled with an antibody (or fragment thereof) or antigen, or receptor or ligand; or enzyme or the enzyme's substrate; or one molecule that binds to another molecule then, respectively, the antigen or antibody (or fragment thereof); or ligand or receptor; substrate or enzyme; or the molecule that another molecule binds to may be used to effect detection and quantitation. Furthermore, the second molecule that binds to the molecule attached to the probe may itself contain a second detectable marker such as an enzyme or isotope. If the label is an enzyme or enzyme inhibitor, then reactions detecting the presence or the absence of enzymatic activity can be utilized. Preferred enzymatic reactions are those which result in a calorimetric change which can be detected utilizing a spectrophotometer. Additional labels include radiopaque substances detected utilizing electromagnetic radiation; magnetic particles detected utilizing magnetic fields; immunological methods involving using antibodies and specific antigens; fluorescent and chemiluminescent markers; and members of any specific binding pair, such as glycolipids or glycoproteins.

To precisely determine the quantity of a target mRNA for ALT2 or ALT1 present in an unknown biological sample, a positive and negative control containing a known quantity of the target nucleic acid sequence can be assayed in parallel with the sample thereby standardizing assay results.

One then compares the amount of mRNA present in the sample to the known amount in normal, healthy tissue. It is preferable that one compares liver tissue to liver tissue and adipose tissue to adipose tissue. If the amount of mRNA present in the sample falls within a pre-determined range, then one diagnoses the person with a certain disease, damage or condition which affects that tissue sampled.

Generation of ALT1 Specific Antibodies and ALT2 Specific Antibodies.

Using the pPET28-ALT1 and pPET28-ALT2 plasmids described above, ALT1 and ALT2 proteins are generated by adding IPTG to the media of the bacteria, as described above. As described above, the bacteria are lysed and the cell-free supernant is collected. This cell-free supernant is applied to a Ni column (HISTRAP, Pharmacia, Peapack, N.J.) equilibrated with a urea containing buffer at a concentration sufficiently high to denature protein. The column is then washed and eluted. Then an aliquot of the eluate is analyzed by gel electrophoresis using antibodies to the polyhistidine tag to confirm the presense of the purified protein.

Purified protein containing fractions of the eluate are dialyzed against an enzyme digestion buffer. The purified protein is injected into rabbits to generate polyclonal antibodies. The rabbits are bled before and 30 days after injection. Samples are stored at −20 ° C. The samples are tested for the presence of polyclonal antibodies by either ELISA or by Western blot with the purified protein. Polyclonal antibodies for ALT1 are assayed for cross-reactivity to ALT2, and polyclonal antibodies for ALT2 are assayed for cross-reactivity to ALT1. Only those animals containing polyclonal antibodies which are specific for one of the enzymes are then sacrificed and splenic cells are isolated. The suspension of splenic cells is mixed with a suspension of HGPRT-negative myeloma cells. Next, 35% glycol is added to the cell suspension, and then the cells are placed in HAT medium which contains hypoxanthine, aminopterin, and thymidine. The hybridoma cells which survive after ten to fourteen days are plated onto 96 well plates.

An alternative approach to generate antibodies specific for ALT1 and specific for ALT2 can also be taken. In this alternative approach, the cDNA encoding ALT2 (SEQ ID NO: 1) is ligated into a pGEX-4T-1 expression vector (Amersham Pharmacia Biotech, Inc., Piscataway, N.J.) such that ALT2 is fused with GST (glutathione-S-transferase). Recombinant protein is expressed in BL21 (DE3) Escherichia coli using 0.8 mM IPTG. Cells are lysed using B-Per reagents (Pierce, Rockford, Ill.); the lysate is incubated with glutathione-agarose beads for 2 hours at 4° C. and subsequently centrifuged for 10 minutes at 3000 rpm. The lysate is washed twice with PBS followed by elution with 10 mM reduced glutathione (Sigma, Co., St. Louis, Mo.) in 50 mM Tris-HCl (pH 7.4).

The resulting purified ALT2-GST is biotinylated using a commercial ECL protein biotinylation kit (Amersharn Pharmacia Biotech. Inc., Piscataway, N.J.). Briefly, 1.0 mg of protein is diluted in 1.0 ml of bicarbonate buffer (pH 8.6), and incubated for 1 hour at room temperature, in 30 μl of biotinylation reagent per mg of protein. After incubation, the protein sample is applied to a Sephadex G25column and eluted with 5.0 ml of PBS (pH 7.4). Fractions of biotinylated ALT2-GST protein are then collected.

The protein profile of the biotinylated ALT2-GST protein is analyzed by 12% (w/v) SDS-PAGE and Silver-Stain procedure (Bio-Rad silver stained plus kit, Bio-Rad, Hercules, Calif.). To determine the presence of ALT2-GST protein, an anti-GST antibody (Amersham Pharmacia Biotech, Inc., Piscataway, N.J.) is used at a 1:1000 dilution, followed by HRP-labeled anti-goat IgG (Santa Cruz Biotechnology, Inc., Santa Cruz, Calif.) at a 1:12,000 dilution in 10% (v/v) blocking buffer. The presence of biotinylated fraction of ALT2-GST protein is detected using streptavidin-horse radish peroxidase conjugated protein (Amersham Pharmacia Biotech, Inc., Piscataway, N.J.) at dilution of 1:6,000 in PBST. Immunodetection is performed using ECL Western Blotting Detection Kit (Amersham Pharmacia Biotech, Inc., Piscataway, N.J.).

Isolated ALT2-GST is injected into guinea pig, rabbit, rat, mouse, or other suitable animal to generate antibodies to ALT2. After multiple dosing, the animal is sacrificed, the spleen isolated, and splenic cells are isolated and hybridoma cells are generated, as described above.

Next the culture media is assayed for the presence of anti-ALT2 antibodies. Biotinylated-ALT2-GST is added to 96-well streptavidin-coated plate. Next, the cell culture from the hybridomas is added to the streptavidin-coated plate. The wells are washed to remove unbound antibody and biotinylated-ALT2-GST. Secondary antibodies specific for the primary antibody and labeled with horse radish peroxidase are added to each well. The wells are washed to remove unbound secondary antibodies and then a substrate solution for horse radish peroxidase is added for fifteen minutes at 25° C. Stop solution of 0.5 M H₂SO₄ is added. Then absorbance is measured at 450 nm. Wells having anti-ALT2 antibodies are determined. The anti-ALT2 antibodies are then applied to an SDS-PAGE gel containing isolated ALT2 to make sure that the antibodies bound to ALT2. Next, the antibodies are applied to an SDS-PAGE gel containing isolated ALT1 to screen for antibodies which are specific for ALT2.

A similar procedure is performed to generate antibodies specific for ALT1 as described above except ALT1 is used instead of ALT2.

If desired monoclonal antibodies are generated to the polyclonal antibodies using well-known in the art techniques.

Synthetic peptide fragments of ALT2 and synthetic peptide fragments of ALT1 can also be used as immunogens for the generation of antibodies.

It is noted that ALT2 polynucleotide sequence can be employed for the construction of recombinant cell lines, recombinant organisms, expression vectors, and the like. Such recombinant constructions can be used to express ALT2 polypeptide. In one embodiment, the recombinant constructions can be used to screen for agents which suppress ALT2 activity or increase ALT2 activity.

When the nucleic acid coding sequences of ALT2 is used for the construction of expression vectors and other types of vectors, the coding sequences of ALT2 is inserted into the coding sequences of the vector, downstream and under the control of a promoter, where the promoter can be inducible, constitutive, or tissue-specific. Additionally, other elements, including regulatory elements, which are commonly found in vectors suitable for use in various molecular biology techniques, can also be included.

In one embodiment, a vector comprising a DNA molecule encoding ALT2 protein is provided. Preferably, a DNA molecule encoding ALT2 of SEQ ID NO:2 is inserted into a suitable expression vector, which is in turn used to transfect or transform a suitable host cell. Exemplary expression vectors include a promoter capable of directing the transcription of a polynucleotide molecule of interest in a host cell. Representative expression vectors include both plasmid and/or viral vector sequences. Suitable vectors include retroviral vectors, vaccinia viral vectors, CMV viral vectors, BLUESCRIPT (Stratagene, San Diego, Calif.) vectors, bacculovirus vectors, and the like. In another embodiment, promoters capable of directing the transcription of a cloned gene or cDNA can be inducible or constitutive promoters and include viral and cellular promoters.

In some embodiments, it can be preferable to use a selectable marker to identify cells that contain the cloned DNA. Selectable markers are generally introduced into the cells along with the cloned DNA molecules and include genes that confer resistance to drugs, such as ampicillin, neomycin, hygromycin, and methotrexate. Selectable markers can also complement auxotrophies in the host cell. Other selectable markers provide detectable signals, such as beta-galactosidase to identify cells containing the cloned DNA molecules.

Antibody Assay

An antibody assay for ALT1 involves taking an aliquot of a sample of fluid (or tissue) and bringing it in contact with ALT1 specific antibodies. Then one measures the amount of ALT1 bound to the antibodies using well-known in the art-field techniques. One then compares that amount of ALT1 bound to the antibody to a known standard for a healthy animal. If the amount of ALT1 in the sample fails within a certain range as compared to the known standard, then one can diagnose the animal with a disease, injury, or condition.

An antibody assay for ALT2 involves taking an aliquot of a sample of fluid (or tissue) and bringing it in contact with ALT2 specific antibodies. Then one measures the amount of ALT2 bound to the antibodies using well-known in the art-field techniques. One then compares that amount of ALT2 bound to the antibody to a known standard for a healthy animal. If the amount of ALT2 in the sample fails within a certain range as compared to the known standard, then one can diagnose the animal with a disease, injury, or condition. Because ALT2 is expressed primarily in the liver, muscle, kidney, brain, and adipose tissue, one can diagnose an animal with liver damage, liver disease, or a conditions and diseases involving liver, muscle, kidney, brain, and adipose tissues.

As an example of an antibody assay, one removes blood from a subject (human, mammal, or animal) and centrifuged for 20 minutes at 4° C. to separate the serum from the rest of the components of blood. Serum is stored at −20° C. until used.

An aliquot of serum is incubated with the ALT1-specific antibodies or ALT2-specific antibodies (as described above) for 1.5 hours at room temperature and then at 4° C. overnight. On the following morning the samples are centrifuged for 20 minutes at 4° C. The resulting precipitate is resuspended in buffer and run on 10% SDS-PAGE. Antibodies specific for the specie ALT1 and ALT2 antibodies are incubated with the gel and analyzed. The advantage of the Western blot analysis is that it can differentiate ALT1 from ALT2 by size as an added measure of specificity.

Because antibodies (or fragments thereof) of the present invention are particularly suited for use in a variety of immunoassays, one can use rapid immunoassay, Western blots, radioimmunoassay, ELISA, immuno-fluorescent microscopy, immuno-electron microscopy, or any other type of immunoassay. The antibodies or the fragment of the antibody can be utilized in liquid phase or bound to a solid-phase carrier.

Antibodies, or fragments thereof, may be labeled using any of a variety of labels and method of labeling. Examples of types of labels which can be used in the present invention include, but are not limited to, enzyme labels, radioisotopic labels, non-radioactive isotopic labels, fluorescent labels, and chemiluminescent labels.

Examples of suitable enzyme labels include, but are not limited to, malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Examples of suitable radioisotopic labels include, but are not limited to, ³H, ¹¹¹In, ¹²⁵I, ³²P, ³³P, ³⁵S, ¹⁴C, ⁵⁷To, ⁵⁸Co, ⁵⁹Fe, ⁷⁵Se, ¹⁵²Eu, ⁹⁰Y, ⁶⁷Cu, ²¹Ci, ²¹¹At, ²¹²Pb, ⁴⁷Sc, and ¹⁰⁹Pd.

Examples of suitable non-radioactive isotopic labels include, but are not limited to, ¹⁵⁷Gd, ⁵⁵Mn, ¹⁶²Dy, ⁵²Tr, and ⁴⁶Fe.

Examples of suitable fluorescent labels include, but are not limited to, a ¹⁵²Eu label, a fluorescein label, an isothiocyanate label, a rhodamine label, a phycoerythrin label, a phycodyanin label, an allophycocyanin label, and a fluorescamine label.

Examples of chemiluminescent labels include, but are not limited to, a luminal label, an isoluminal label, an aromatic acridinium ester label, an imidazole label, an acridinium salt label, an oxalate ester label, a luciferin label, and a luciferase label.

Those of ordinary skill in the art will know of other suitable labels which may be employed in accordance with the present invention. The binding of these labels to antibodies or fragments thereof can be accomplished using standard techniques commonly known to those of ordinary skill in the art. Typical techniques are described by Kennedy, J. H., et al., Protein-protein coupling reactions and the applications of protein conjugates, Clin. Chim Acta, 70:1-31 (1976), and Schuurs and Van Weemen, Enzyme-imnuunoassay, Clin. Chim Acta, 81:1-40 (1977). Coupling techniques mentioned in the latter are the glutaraldehyde method, the periodate method, the dimaleimide method, and others, all of which are incorporated by reference herein.

The detection of the antibodies (or fragments of antibodies) of the present invention can be improved through the use of carriers. Well-known carriers include glass, polystyrene, polypropylene, polyethylene, dextran, nylon, amylases, natural and modified celluloses, polyacrylamides, agaroses, and magnetite. The nature of the carrier can be either soluble to some extent or insoluble for the purposes of the present invention. The support material may have virtually any possible structural configuration so long as the coupled molecule is capable of binding to ALT2 or ALT1. Thus, the support configuration may be spherical, as in a bead, or cylindrical, as in the inside surface of a test tube, or the external surface of a rod. Alternatively, the surface may be flat, such as a sheet, or test strip. Those skilled in the art will note many other suitable carriers for binding monoclonal antibody, or will be able to ascertain the same by use of routine experimentation.

The antibodies, or fragments of antibodies of ALT2 or ALT1 may be used to quantitatively or qualitatively detect the presence of ALT2 or ALT 1. Such detection may be accomplished using any of a variety of immunoassays known to persons of ordinary skill in the art such as radioimmunoassays, immunometic assays, immuno-PCR, and Western blot. Using standard methodology well known in the art, a diagnostic assay can be constructed by coating on a surface (i.e. a solid support) for example, a microtitration plate or a membrane (e.g. nitrocellulose membrane), antibodies specific for ALT2 or a portion of ALT2, and contacting it with a sample of bodily fluid such as serum from an animal suspected of having damage or a disease affecting tissue that produces ALT2. The presence of a resulting complex formed between ALT2 in the serum and ALT2-specific antibodies can be detected by any of the known detection methods common in the art such as fluorescent antibody spectroscopy or colorimetry. A good description of a radioimmune assay may be found in Laboratory Techniques and Biochemistry in Molecular-Biology. by Work, T. S., et al. North Holland Publishing Company, N.Y. (1978), incorporated by reference herein. Sandwich assays are described by Wide at pages 199-206 of Radioimmune Assay Method, edited by Kirkham and Hunter, E. & S. Livingstone, Edinburgh, 1970.

One can also use antibodies to ALT1 and ALT2 to follow the progression of liver damage or other conditions in a subject. One can perform assays over time for ALT1 and/or ALT2 and quantify the improvement or worsening of the condition or damage by comparing the levels of the ALT enzymes over time

Kits

The methods described above may be practiced with the kits of this invention for detection of ALT2 and/or ALT1 in an animal using ALT1 specific antibodies and/or ALT2 specific antibodies described above. The kits for the detection of ALT1 and/or ALT2 may contain, for example, the antibodies described above, monoclonal or polyclonal, or their fragments, which selectively bind to ALT1 or ALT2, respectively, or a portion therof, which may be conjugated to a fluorochrome, a phosphochrome, enzyme, or radiolabel, an appropriate substrate for enzyme-linked antibodies, e.g., hydrogen peroxide for a peroxidase, blocking solutions, e.g., normal goat or rabbit serum, 3% bovine serum albumin in physiological saline, and the like, and buffers, e.g., Tris-HCl, phosphate buffered saline, EDTA, and the like, among others, as well as combinations of any two or more thereof. The kit may have one or more containers which contain the reagents. It is preferable, but not necessary, that the ALT 1-specific antibodies be in one container and that the ALT2-specific antibodies be in a second container. Other containers can contain the reagents necessary for the determination of binding of ALT1 or ALT2 to the antibodies. Use of the kit is accomplished by adding a sample which contains ALT1 and/or ALT2 to one of the containers and allowing the sample to contact ALT 1-specific antibodies or ALT2-specific antibodies for a suitable amount of time. After which, the container may be washed and a labeled antibody which is specific for ALT1 or specific ALT2 is added to the desired container and then the container is washed and the amount of labeled antibody is determined. The kit may have instructions on determining the amount of ALT1 and/or ALT2 present in a sample and the type of diagnosis for which those levels are indicative. The kit may also have negative and positive controls.

The examples provided herein are for illustrative purposes only and are in no way intended to limit the scope of the present invention. While the invention has been described in detail, and with reference to specific embodiments thereof, it will be apparent to one with ordinary skill in the art that various changes and modifications can be made therein without departing from the spirit and scope thereof.

1-7. (canceled)

8. A method of diagnosing or detecting injury or disease, the method comprising:
a. contacting a sample of a bodily fluid from an animal with at least one first antibody, wherein said first antibody specifically binds to a first polypeptide, wherein said first polypeptide comprises an amino acid sequence of SEQ ID NO:2 or an amino acid sequence having at least 95% homology to the amino acid sequence of SEQ ID NO:2;
b. detecting said first antibody which is bound to said first polypeptide in said sample;
c. contacting said sample with at least one second antibody, wherein said second antibody specifically binds to a second polypeptide, wherein said second polypeptide comprises an amino acid sequence having at least 95% homology to the amino acid sequence of SEQ ID NO:3;
d. detecting said second antibody which is bound to said second polypeptide in said sample; and
e. comparing the amount of said first polypeptide bound to said first antibody and the amount of said second polypeptide bound to said second antibody, wherein when the amount of said bound first polypeptide being sufficiently higher than the amount of said bound second polypeptide indicates said animal has said disease or injury.

9. The method of claim 8 wherein said bodily fluid is selected from a group comprising blood, serum, lymph, urine, sweat, mucus, sputum, saliva, semen, spinal fluid, interstitial fluid, synovial fluid, cerebrospinal fluid, gingival fluid, vaginal fluid, and pleural fluid.

10. The method of claim 8 wherein said injury or disease is in a tissue selected from a group comprising liver, brain, muscle, adipose tissue, and kidney.

11. A method of diagnosing or detecting injury or disease, the method comprising:
a. contacting a sample of a bodily fluid from an animal suspected of having said injury or disease with at least one first antibody, wherein said first antibody specifically binds to a first polypeptide, wherein said first polypeptide comprises an amino acid sequence of SEQ ID NO:2 or an amino acid sequence having 95% homology to the amino acid sequence of SEQ ID NO:2;
b. detecting said first antibody which is bound to said first polypeptide in said sample; and
c. comparing said amount of said first polypeptide in said sample to the amount of said first polypeptide in the bodily fluid of a control animal, wherein the amount of said first polypeptide in said sample being higher than the amount of said first polypeptide in the bodily fluid of the control animal indicates that said animal has said disease or injury.

12. The method of claim 11 wherein said bodily fluid is selected from a group comprising blood, serum, lymph, urine, sweat, mucus, sputum, saliva, semen, spinal fluid, interstitial fluid, synovial fluid, cerebrospinal fluid, gingival fluid, vaginal fluid, and pleural fluid.

13. The method of claim 11 wherein said injury or disease is in a tissue selected from a group comprising liver, brain, muscle, adipose tissue, and kidney.

14. A diagnostic kit for use in diagnosing damage or disease, the kit comprising:
a. a measurer of levels of a first polypeptide in a sample of a bodily fluid, wherein the first polypeptide comprises an amino acid sequence of SEQ ID NO:2 or an amino acid sequence having 95% homology to the amino acid sequence of SEQ ID NO:2; and
b. an indicator for determining if a measurement from the measurer of step (a) falls in a range associated with said damage or disease.

15. The diagnostic kit of claim 14 further comprising:
c. a measurer of levels of a second polypeptide in a sample of a bodily fluid, wherein said second polypeptide comprises an amino acid sequence of SEQ ID NO:3 or an amino acid sequence having at least 95% homology to the amino acid sequence of SEQ ID NO:3; and
d. an indicator for determining if a measurement from the measurer of step (c) falls in a range associated with damage or disease.

16. The kit of claim 14 wherein said measurer is selected from the group comprising a biologic assay, an antibody-based assay, an enzyme linked immunosorbent assay, a Western blot, a rapid immunoassay, and a radioimmunoassay.

17. A diagnostic kit for use in diagnosing damage or disease, the kit comprising;
a. an antibody that binds specifically to a first polypeptide comprising the amino acid sequence of SEQ ID NO:2 or an amino acid sequence having 95% homology to the amino acid sequence of SEQ ID NO:2;
b. immunoassay reagents; and
c. a control for determining if a measurement of the first polypeptide indicates a diagnosis of damage or disease.

18. The kit of claim 17 wherein said control comprises instructions indicating that an increase or decrease in the amount of the first polypeptide indicates a diagnosis for damage or disease.

19. The kit of claim 17 further comprising;
a. an antibody that binds specifically to a second polypeptide, wherein said second polypeptide comprises an amino acid sequence of SEQ ID NO:3 or an amino acid sequence having at least 95% homology to the amino acid sequence of SEQ ID NO:3;
b. a control for determining if a measurement of the second polypeptide indicates a diagnosis of damage or disease.

20. The kit of claim 19 wherein said control comprises instructions indicating that an increase or decrease in the amount of the second polypeptide indicates a diagnosis for damage or disease.

21. A diagnostic kit for use in a condition associated with altered levels of an alanine transaminase (ALT) polypeptide in a bodily fluid, the kit comprising:
a. at least one measurer of levels of the alanine transaminase polypeptide in a sample of the bodily fluid, wherein the alanine transaminase comprises an amino acid sequence of SEQ ID NO:2, an amino acid sequence having at least 95% homology to the amino acid sequence of SEQ ID NO:2, an amino acid sequence of SEQ ID NO:3, an amino acid sequence having 95% homology to the amino acid sequence of SEQ ID NO:3, or combinations thereof; and
b. an indicator for determining if a measurement from the at least one measurer of step (a) falls in a range associated with said condition.

22-23. (canceled)

24. A method of diagnosing an adverse condition in an animal, the method comprising:
a. contacting a sample of a bodily fluid from said animal with at least one antibody, wherein said antibody specifically binds to a first polypeptide, wherein said first polypeptide comprises an comprising the amino acid sequence of SEQ ID NO:2 or an amino acid sequence having at least 95% homology to the amino acid sequence of SEQ ID NO:2; and
b. detecting said antibody which is bound to said first polypeptide in said sample; and
c. comparing said amount of said detected antibody to a known quantity for a control animal, wherein an increased amount of detected antibody in the animal as compared to the control animal diagnoses the animal with the adverse condition.

25. (canceled)

26. The method of claim 24 wherein said bodily fluid is selected from a group comprising blood, serum, lymph, urine, sweat, mucus, sputum, saliva, semen, spinal fluid, interstitial fluid, synovial fluid, cerebrospinal fluid, gingival fluid, vaginal fluid, and pleural fluid.

27. The kit of claim 21, wherein said measurer is selected from the group comprising a biologic assay, an antibody-based assay, an enzyme linked immunosorbent assay, a Western blot, a rapid immunoassay, and a radioimmunoassay.

28-29. (canceled)


SEQUENCE LISTING (37 sequences)

SEQ ID NO: 1 - DNA, 3768 bp, Homo sapiens
  ref <301>: AUTHORS: Yang. R.Z., Blaileanu, G., Hansen, B.C., Shuldiner, A.R. and Gong, D.W.
  ref <302>: TITLE: cDNA Cloning, Genomic Structure, Chromosomal Mapping, and Functiona1 Expression of Novel Human Alanine Aminotransferase
  ref <303>: JOURNAL: Genomics
  ref <304>: VOLUME: 79
  ref <305>: ISSUE: 3
  ref <306>: PAGES: 445-450
  ref <307>: DATE: 2002
  ref <308>: DATABASE ACCESSION NUMBER: GenBank/AY029173
  ref <309>: DATABASE ENTRY DATE: 2001-04-16
ggcggtgctcaaggtgcggcccgagcgcagccggcgcgagcgcatcctcacgctggagtccatgaacccgcaggtgaaggcggtggagtacgccgtgcggggacccatcgtgctcaaggccggcgagatcgagctcgagctgcagcggggtatcaaaaagccattcacagaggtcatccgagccaacatcggggacgcccaggctatggggcagcagccaatcaccttcctccggcaggtgatggcactatgcacctacccaaacctgctggacagccccagcttcccagaagatgctaagaaacgtgcccggcggatcctgcaggcttgtggcgggaacagcctggggtcctacagtgctagccagggtgtcaactgcatccgtgaagatgtggctgcctacatcaccaggagggatggcggtgtgcctgcggaccccgacaacatctacctgaccacgggagctagtgacggcatttctacgatcctgaagatcctcgtctccgggggcggcaagtcacggacaggtgtgatgatccccatcccacaatatcccctctattcagctgtcatctctgagctcgacgccatccaggtgaattactacctggacgaggagaactgctgggcgctgaatgtgaatgagctccggcgggcggtgcaggaggccaaagaccactgtgatcctaaggtgctctgcataatcaaccctgggaaccccacaggccaggtacaaagcagaaagtgcatagaagatgtgatccactttgcctgggaagagaagctctttctcctggctgatgaggtgtaccaggacaacgtgtactctccagattgcagattccactccttcaagaaggtgctgtacgagatggggcccgagtactccagcaacgtggagctcgcctccttccactccacctccaagggctacatgggcgagtgtggttacagaggaggctacatggaggtgatcaacctgcaccctgagatcaagggccagctggtgaagctgctgtcggtgcgcctgtgccccccagtgtctgggcaggccgccatggacattgtcgtgaaccccccggtggcaggagaggagtcctttgagcaattcagccgagagaaggagtcggtcctgggtaatctggccaaaaaagcaaagctgacggaagacctgtttaaccaagtcccaggaattcactgcaaccccttgcagggggccatgtacgccttccctcggatcttcattcctgccaaagctgtggaggctgctcaggcccatcaaatggctccagacatgttctactgcatgaagctcctggaggagactggcatctgtgtcgtgcccggcagtggctttgggcagagggaaggcacttaccacttcaggatgactatcctccctccagtggagaagctgaaaacggtgctgcagaaggtgaaagacttccacatcaacttcctggagaagtacgcgtgaggacgcctgagccccagcgggagacctgtccttggctcttcctcccaatgcccgtcaggctgaactcgcctcccccgtgactctgcctcgggcctcgcagaggccgctggtcacttcgtcatcattttgcccctggagacgtctttctttgtgccttgatgttgagagcgcctctcttttgagcaaacaagcattctatatgcaaccagagtagaggggacctgctcagcaggtgtgaccagggttctctgaatctgttattgtttttgcttctggaaagttcatttggggtttacaacaactaggatgtgttgggtgagatgtttcagatctggagaaatgagcaggtgtcgggaaatgtgtgacttaaccgtggtgagggctggaaatccaaactcaccaccatgatctgtgaaataaagcccttagcggtgtgaagcatccggtcctttgaacagaagggcctggaaggcccctggggctgagaaagggtccgcccggtggcctggaggcaggcgccgggagcgcagtagcacgtggactgggcaggatgttgcactagcttggggtagatgctgggggctgcggccacggtcagagggccccactgtgaggcgtgggtgtgagccaggctgcaggaggaactgggcctccgcttcccagcaacgcagccaggcctgagaattctgtgcgcccggcgggctttgggaatgaggggttcccttgaacatgcgtaggctggaaccccgtctgagaggtctccctgaatttcagtgacacatagtgcagcccggcagtgtcccacttccgtggagagagccgctggaatggtgtggacccatcccgcgggtgaccggtgcctgttctcccctgaccgagcctgtgagcacatcgccccctgctggcgacagcggggaaatgagggctgaaaatatcctccccacaagggcaatccccgggacctgccgagcagccaaggccctgtcctttcttgaatggtggcgagctgaatctggtcggtttcctagcttttaggtggtaaaagtgcctggcagcttggctgccgtggaggagtcagtcgtggttggaggttcattgccgtgctttcatgcagagtgttttgccttcatgttagcttccggctcccctcccaggctgcagactctgacctgtggcatcaggcttctcccagtacaggagggtgccatcccccagcatgcggcttctctgccattagcagccctgggcgggccgaccacactcgaggctgcggtgctacgggcttagccctcgcctccctcactgggagcttccccatcctccctgccttccccagtgggaagttagggaagctcaggagcctgggaccccgcatgtcccaaaatgggattggagaagctggagagaaagcagaagaggccgaggagtgaggcagcagcctctatgcttgatttccacaccgggtccgtgcagaggaaacagaaactcccaactgtccttacccaccgacatcacagcccctatgaagaaagtagccacaatctcaaataacaaaagggaatgttctaaaactttttcttccttaaaaaatggagaaaattgcacttgtgcttgctgtgtggtatataaaccaggattagtcccagggtcgtgaggtttctggtgaaaaggttaaatcgtagaagctagtatattttttatatttttgtaacaattgcttttttcatgggggaggcggggttagtatttatagtcctaacaagtccagtaattttttataaatcttcagattataaacagcccctaaaaactttacaacgtttacacagttttttaaaaagagactgtatacacttgatttgctttcaaaataaataaggtcagctagtctaggaggttaacgtcgggtaggaatgctgatcatgataggtttggttttctacagattctgttccggtgcctttcctatccaggcaccacctgagaaagttgtcatttgaggtcgcacttggaagttacatctgtgaagtttctgtcattcgtccagatctgtgtgtgtagcatgtgctgaggaagcacgtgctgggctgtgcctcagacagtgcatcaccgggcacccagaggcttgcctggctattcctgttctggtgtgtgtggagtgttggggaggaacagatgcagatcaacctgtggctgttttcccgtctaggttctcacaggtatctcctgacagaggtacttaacaatggctctgctggaaatttctataaataaaatgtccaaaatggaaa

SEQ ID NO: 2 - protein, 523 aa, Homo sapiens
  ref <301>: AUTHORS: Yang, R.Z., Blaileanu, G., Hansen, B.C., Shuldiner, A.R. and Gong, D.W.
  ref <302>: TITLE: cDNA Cloning, Genomic Structure, Chromosomal Mapping, and Functiona1 Expression of Novel Human Alanine Aminotransferase
  ref <303>: JOURNAL: Genomics
  ref <304>: VOLUME: 79
  ref <305>: ISSUE: 3
  ref <306>: PAGES: 445-450
  ref <307>: DATE: 2002
  ref <308>: DATABASE ACCESSION NUMBER: GenBank/AY029173
  ref <309>: DATABASE ENTRY DATE: 2001-04-16
MQRAAALVRRGCGPRTPSSWGRSQSSAAAEASAVLKVRPERSRRERILTLESMNPQVKAVEYAVRGPIVLKAGEIELELQRGIKKPFTEVIRANIGDAQAMGQQPITFLRQVMALCTYPNLLDSPSFPEDAKKRARRILQACGGNSLGSYSASQGVNCIREDVAAYITRRDGGVPADPDNIYLTTGASDGISTILKILVSGGGKSRTGVMIPIPQYPLYSAVISELDAIQVNYYLDEENCWALNVNELRRAVQEAKDHCDPKVLCIINPGNPTGQVQSRKCIEDVIHFAWEEKLFLLADEVYQDNVYSPDCRFHSFKKVLYEMGPEYSSNVELASFHSTSKGYMGECGYRGGYMEVINLHPEIKGQLVKLLSVRLCPPVSGQAAMDIVVNPPVAGEESFEQFSREKESVLGNLAKKAKLTEDLFNQVPGIHCNPLQGAMYAFPRIFIPAKAVEAAQAHQMAPDMFYCMKLLEETGICVVPGSGFGQREGTYHFRMTILPPVEKLKTVLQKVKDFHINFLEKYA

SEQ ID NO: 3 - protein, 496 aa, Homo sapiens
  ref <301>: AUTHORS: Sohocki, M.M., Sullivan, L.S., Harrison, W.R., Sodergren, E.J., Elder, F.F., Weinstock, G., Tanase, S. and Daiger, S.P.
  ref <302>: TITLE: Human Glutamate Pyruvate Transaminase (GPT): Localization to 8q24.3, cDNA and Genomic Sequences, and Polymorphic Sites
  ref <303>: JOURNAL: Genomics
  ref <304>: VOLUME: 40
  ref <305>: ISSUE: 2
  ref <306>: PAGES: 247-252
  ref <307>: DATE: 1997
  ref <308>: DATABASE ACCESSION NUMBER: GenBank/NM_005309
  ref <309>: DATABASE ENTRY DATE: 2000-11-01
MASSTGDRSQAVRHGLRAKVLTLDGMNPRVRRVEYAVRGPIVQRALELEQELRQGVKKPFTEVIRANIGDAQAMGQRPITFLRQVLALCVNPDLLSSPNFPDDAKKRAERILQACGGHSLGAYSVSSGIQLIREDVARYIERRDGGIPADPNNVFLSTGASDAIVTVLKLLVAGEGHTRTGVLIPIPQYPLYSATLAELGAVQVDYYLDEERAWALDVAELARALGQARDHCRPRALCVINPGNPTGQVQTRECIEAVIRFAFEERLFLLADEVYQDNVYAAGSQFHSFKKVLMEMGPPYAGQQELASFHSTSKGYMGECGFRGGYVEVVNMDAAVQQQMLKLMSVRLCPPVPGQALLDLVVSPPAPTDPSFAQFQAEKQAVLAELAAKAKLTEQVFNEAPGISCNPVQGAMYSFPRVQLPPRAVERAQELGLAPDMFFCLRLLEETGICVVPGSGFGQREGTYHFRMTILPPLEKLRLLLEKLSRFHAKFTLEYS

SEQ ID NO: 4 - DNA, 27 bp, Homo sapiens
  primer_bind (1)..(27): Primer P922 from TripEX vector
aatacgactcactatagggcgaattgg

SEQ ID NO: 5 - DNA, 26 bp, Homo sapiens
  primer_bind (1)..(26): Primer P923 from TripEX vector
ctcgggaagcgcgccattgtgttggt

SEQ ID NO: 6 - DNA, 18 bp, Homo sapiens
  primer_bind (1)..(18): Primer P927 from TripEX vector
gttggtacccgggaattc

SEQ ID NO: 7 - DNA, 20 bp, Homo sapiens
  primer_bind (1)..(20): ALT2-specific primer p1106 [nucleotide (nt) 447-428 in AY029173]
taggtgcatagtgccatcac

SEQ ID NO: 8 - DNA, 22 bp, Homo sapiens
  primer_bind (1)..(22): Primer P1145 [(nt) 425-446 in AY029173]
caggtgatggcactatgcacct

SEQ ID NO: 9 - DNA, 24 bp, Homo sapiens
  primer_bind (1)..(24): Primer P1090 [(nt) 653-630 in AY029173]
ctcccgtcctcaggtagatgttgt

SEQ ID NO: 10 - DNA, 12 bp, Homo sapiens
  5′clip (1)..(12): ALT2 Exon 1 exon/intron junction, 5′ splice donor
cgacaggcacgt

SEQ ID NO: 11 - DNA, 12 bp, Homo sapiens
  3′clip (1)..(12): ALT2 Exon 1 Intron/Exon junction, 3′ splice acceptor
tgccagggtttc

SEQ ID NO: 12 - DNA, 12 bp, Homo sapiens
  5′clip (1)..(12): ALT2 Exon 2 exon/intron junction, 5′ splice donor
cagcgggtgagc

SEQ ID NO: 13 - DNA, 12 bp, Homo sapiens
  3′clip (1)..(12): ALT2 Exon 2, intron/exon junction 3′ splice acceptor
gcccagggtatc

SEQ ID NO: 14 - DNA, 12 bp, Homo sapiens
  5′clip (1)..(12): ALT2 Exon 3 exon/intron junction 5′ splice donor
cggcaggtgagc

SEQ ID NO: 15 - DNA, 12 bp, Homo sapiens
  3′clip (1)..(12): ALT2 Exon 3, intron/exon junction, 3′ splice acceptor
ccccaggtgatg

SEQ ID NO: 16 - DNA, 12 bp, Homo sapiens
  5′clip (1)..(12): ALT2 Exon 4, exon/intron junction, 5′ splice donor
gcctgggtgagg

SEQ ID NO: 17 - DNA, 12 bp, Homo sapiens
  3′clip (1)..(12): ALT2 Exon 4 intron/exon junction, 3′ splice acceptor
ttacagggtcct

SEQ ID NO: 18 - DNA, 12 bp, Homo sapiens
  5′clip (1)..(12): ALT2 Exon 5, exon/intron junction, 5′ splice donor
atttctgtacgt

SEQ ID NO: 19 - DNA, 12 bp, Homo sapiens
  3′clip (1)..(12): ALT2 Exon 4, intron/exon junction, 3′ splice acceptor
ttgcagacgatc

SEQ ID NO: 20 - DNA, 12 bp, Homo sapiens
  5′clip (1)..(12): ALT2 Exon 6, exon/intron junction, 5′ splice donor
ccacaggtctgc

SEQ ID NO: 21 - DNA, 12 bp, Homo sapiens
  3′clip (1)..(12): ALT2 Exon 6, intron/exon junction, 3′ splice acceptor
ttataggccagg

SEQ ID NO: 22 - DNA, 12 bp, Homo sapiens
  5′clip (1)..(12): ALT2 Exon 7, exon/intron junction, 5′ splice donor
gatgaggtaaga

SEQ ID NO: 23 - DNA, 12 bp, Homo sapiens
  3′clip (1)..(12): ALT2 Exon 7 intron/exon junction, 3′ splice donor
ccgcaggtgtac

SEQ ID NO: 24 - DNA, 12 bp, Homo sapiens
  5′clip (1)..(12): ALT2 Exon 8, exon/intron junction, 5′ splice donor
gggcgagtacgt

SEQ ID NO: 25 - DNA, 12 bp, Homo sapiens
  3′clip (1)..(12): ALT2 Exon 8, intron/exon junction 3′ splice acceptor
ctccaggtgtgg

SEQ ID NO: 26 - DNA, 12 bp, Homo sapiens
  5′clip (1)..(12): ALT2 Exon 9 exon/intron junction, 5′ splice donor
agccgagtgagt

SEQ ID NO: 27 - DNA, 12 bp, Homo sapiens
  3′clip (1)..(12): ALT2 Exon 9, intron/exon junction, 3′ splice acceptor
catcaggagaag

SEQ ID NO: 28 - DNA, 12 bp, Homo sapiens
  5′clip (1)..(12): ALT2 Exon 10, exon/intron junction, 5′ splice donor
gctcaggtctgg

SEQ ID NO: 29 - DNA, 12 bp, Homo sapiens
  3′clip (1)..(12): ALT2 Exon 10 intron/exon junction, 3′ splice acceptor
ccataggcccat

SEQ ID NO: 30 - DNA, 12 bp, Homo sapiens
  5′clip (1)..(12): ALT2 Exon 11 exon/intron junction, 5′ splice donor
cttcaggtatga

SEQ ID NO: 31 - DNA, 12 bp, Homo sapiens
  3′clip (1)..(12): ALT2 Exon 11 intron/exon junction, 3′ splice acceptor
tgccaggatgac

SEQ ID NO: 32 - DNA, 21 bp, Homo sapiens
  primer_bind (1)..(21): ALT2 primer for chomosomal localization
ggcaggatgttgcactagctt

SEQ ID NO: 33 - DNA, 21 bp, Homo sapiens
  primer_bind (1)..(21): ALT2 primer for chromosomal localization
ggctgcactatgtgtcactga

SEQ ID NO: 34 - DNA, 32 bp, Homo sapiens
  primer_bind (1)..(32): NdeI-linked primer P1266 for ALT2 (nt 95 to 114, AY029173)
acctgaattcatatgcagcgggcggcggcgct

SEQ ID NO: 35 - DNA, 34 bp, Homo sapiens
  primer_bind (1)..(34): HindIII-linked primer p1117 for ALT2 (nt 1666 to 1646, AY029173)
ggctcagaagctttcacgcgtacttctccagcaa

SEQ ID NO: 36 - DNA, 33 bp, Homo sapiens
  primer_bind (1)..(33): Primer p1118 for ALT1 (nt 268 to 288, NM_005309)
ctgggtagacatatggcctcgagcacaggtgac

SEQ ID NO: 37 - DNA, 37 bp, Homo sapiens
  primer_bind (1)..(37): Primer p1119 for ALT1 (nt 1158 to 1137, NM_005309)
ccccagctgaagctttcaggagtactcgagggtgaac